(19) United States
(12) Patent Application Publication
(10) Pub. No.: US 2026/0262999 A1
(43) Pub. Date: Sep. 10, 2026

(54) METHOD AND SYSTEM OF MONITORING ANESTHESIA DEPTH

(71) Applicant: The Board of Regents, The University of Texas System, Austin, TX (US)

(72) Inventors: Yandong Jiang, Houston, TX (US); Xiaoqian Jiang, Houston, TX (US)

(21) Appl. No.: 19/166,212

(22) PCT Filed: Mar. 19, 2024
(86) PCT No.: PCT/US2024/020528

§ 371 Date: Sep. 17, 2025

Related U.S. Application Data
(60) Provisional application No. 63/453,596, filed on Mar. 21, 2023.

(51) Int. Cl. A61B 5/00; A61B 5/31; A61B 5/372; A61B 5/38

(52) U.S. Cl. CPC A61B 5/4821; A61B 5/31; A61B 5/372; A61B 5/38; A61B 5/7267

(57) ABSTRACT

Embodiments of the present disclosure provide systems and methods for anesthesia depth monitoring. One such method comprises obtaining baseline EEG data of a subject prior to sedation; obtaining time-series EEG data of the subject after initiation of sedation; identifying loss of consciousness (LOC) of the subject; generating profiles segmented from the time-series EEG data that correspond to the LOC of the subject and sedation levels of the subject before and after LOC; training an AI sedation model associated with the subject using the generated profiles, wherein the AI sedation model is trained to determine a sedation level of the subject based upon a profile segmented from the time-series EEG data of the subject; determining a current sedation level of the subject based upon a current profile segmented from the time-series EEG data; and/or providing an indication of a comparison of the current sedation level with a target sedation level.

### CROSS-REFERENCE TO RELATED APPLICATION

This application claims priority to co-pending U.S. provisional application entitled, “Method and System of Monitoring Anesthesia Depth,” having application No. 63/453,596, filed Mar. 21, 2023, which is entirely incorporated herein by reference.

### TECHNICAL FIELD

The present invention is generally related to intraoperative monitoring.

### BACKGROUND

With the current practice of intraoperative monitoring, the median rate of intraoperative awareness is estimated to be 37% and the rate of awareness with post operation recall is 0.1-0.4% under general anesthesia. It was estimated 20,000 to 40,000 individuals experience intraoperative awareness annually in the United States alone. Due to the fear of intraoperative awareness and its catastrophic consequences, clinicians often over-sedate the patients under general anesthesia. To avoid the risk of a small fraction of patients experiencing intraoperative awareness and postoperative recall secondary to undersedation, most patients are exposed to oversedation. However, oversedation also imposes risks to the patients, as it is associated with long-term (>1 year) mortality. This is particularly significant for the elderly population. Therefore, such a current practice renders a frequent occurrence of inappropriate sedation, either over or under, of the patients under general anesthesia.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of a system for monitoring patient variables, in accordance with various embodiments of the present disclosure.

FIG. 2 illustrates an example of a closed-loop system for anesthesia delivery to a patient, in accordance with various embodiments of the present disclosure.

FIG. 3 illustrate an example of electroencephalogram (EEG) profiles and EEG readings versus time during general anesthesia, in accordance with various embodiments of the present disclosure.

FIG. 4 is a flow chart illustrating an example of the monitoring of anesthesia depth, in accordance with various embodiments of the present disclosure.

FIG. 5 is a flow chart illustrating an example of interactively monitoring sedation level via a brain-computer interface, in accordance with various embodiments of the present disclosure.

FIG. 6 is a schematic block diagram of an example of a computing device that can be utilized for monitoring anesthesia depth, in accordance with various embodiments of the present disclosure.

### SUMMARY

Embodiments of the present disclosure provide a system and method for anesthesia depth monitoring. One such method comprises obtaining baseline electroencephalogram (EEG) data of a subject prior to sedation; obtaining time-series EEG data of the subject after initiation of sedation; identifying loss of consciousness (LOC) of the subject; generating profiles segmented from the time-series EEG data, the profiles corresponding to the LOC of the subject and a plurality of sedation levels of the subject before and after LOC, where the plurality of sedation levels comprise a target sedation level for the subject; training an artificial intelligence (AI) sedation model associated with the subject using the generated profiles, the AI sedation model trained to determine a sedation level of the subject based upon a profile segmented from the time-series EEG data of the subject; determining a current sedation level of the subject based upon a current profile segmented from the time-series EEG data of the subject; and/or providing an indication of a comparison of the current sedation level with the target sedation level.

The present disclosure can also be viewed as a system for anesthesia depth monitoring. In this regard, such a system comprises monitoring apparatus configured to provide EEG data of a subject; and a computing device comprising processing circuitry configured to obtain baseline EEG data of the subject from the monitoring apparatus prior to sedation and time-series EEG data of the subject after initiation of sedation; generate profiles segmented from the time-series EEG data, the profiles corresponding to an identified loss of consciousness (LOC) of the subject and a plurality of sedation levels of the subject before and after the LOC, where the plurality of sedation levels comprise a target sedation level for the subject; train an AI sedation model associated with the subject using the generated profiles, the AI sedation model trained to determine a sedation level of the subject based upon a profile segmented from the time-series EEG data of the subject; determine a current sedation level of the subject based upon a current profile segmented from the time-series EEG data of the subject; and/or provide an indication of a comparison of the current sedation level with the target sedation level.

In one or more aspects for such systems and/or methods, the plurality of sedation levels comprise undersedation and oversedation levels; the undersedation levels comprise light sedation, moderate sedation, and heavy sedation levels; the identification of the LOC comprises monitoring the subject's responsiveness to a rhythmic stimulus, wherein the LOC is identified based on a lack of a response to the rhythmic stimulus; the rhythmic stimulus is a pre-recorded audio command provided by the computing device; monitoring responsiveness comprises detecting a physical response provided by the subject in response to the rhythmic stimulus; monitoring responsiveness comprises detecting an EEG profile generated by the subject in response to the rhythmic stimulus; the baseline EEG data comprises EEG data obtained with the subject's eyes open and closed; the plurality of sedation levels comprise sedation levels at two minutes before LOC, one minute before LOC, one minute after LOC, and two minutes after LOC; the target sedation level corresponds to the sedation level one minute after LOC; the AI sedation model comprises a residual neural network; the processing circuitry is further configured to detect an EEG profile generated by the subject in response to a verbal command, the EEG profile indicating a current condition of the subject; providing the indication of the comparison comprises providing the current sedation level with the target sedation level for display on a monitor; and/or the processing circuitry is configured to provide an adjustment to the sedation based upon the comparison of the current sedation level with the target sedation level.

In one or more aspects, such systems and/or methods involve or comprise a sensor configured to detect grip pressure applied in response to the rhythmic stimulus.

Other systems, methods, features, and advantages of the present disclosure will be or become apparent to one with skill in the art upon examination of the following drawings and detailed description. It is intended that all such additional systems, methods, features, and advantages be included within this description and be within the scope of the present disclosure.

### DETAILED DESCRIPTION

Disclosed herein are various examples related to anesthesia depth monitoring and control. Reference will now be made in detail to the description of the embodiments as illustrated in the drawings, wherein like reference numbers indicate like parts throughout the several views.

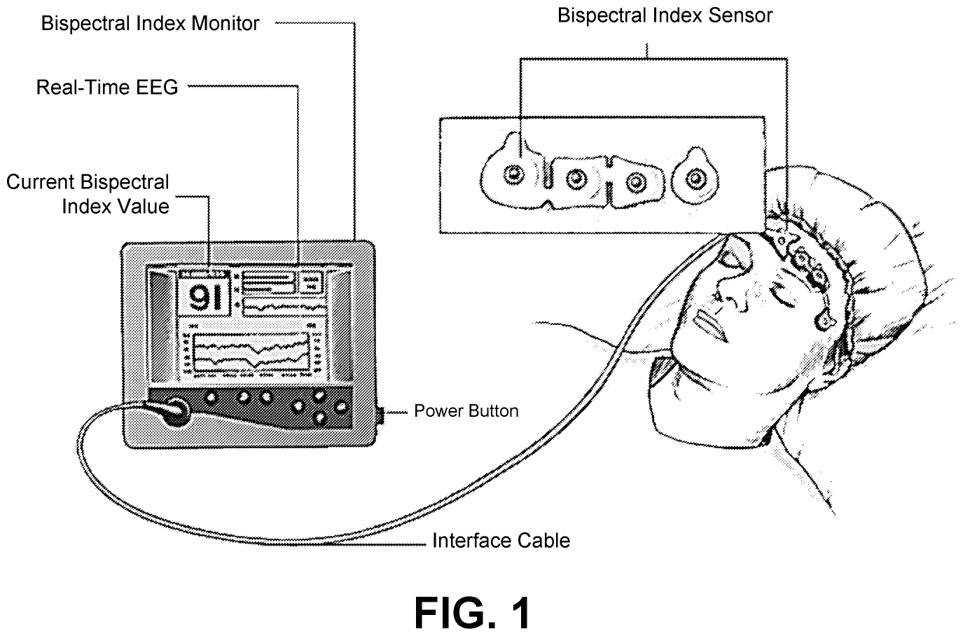

As noted by G. A. Mashour et al. (“Intraoperative awareness: controversies and non-controversies” British Journal of Anaesthesia, Vol. 115, Suppl 1, pp. i20-i26, July 2015), the unintended experience and memory of surgical or procedural events can be devastating for patients and remains a dynamic area of investigation. Intraoperative awareness, with or without explicit episodic recall, is relevant to patient safety, standards for intraoperative monitoring, and the search for the neural correlates of consciousness. FIG. 1 illustrates an example of a system for monitoring patient variables, e.g., electroencephalogram (EEG) and Bispectral (BIS) Index. While the measurement of sedation level during general anesthesia and sedation is important to the success of a procedure, it is a challenge to reliably monitor the level of sedation due to patient heterogeneity. The techniques currently in use are based on processed EEG using a population-based approach. Details of current techniques can be found in, e.g., U.S. Pat. No. 10,542,903 (“Depth of Consciousness Monitor”) issued Jan. 28, 2020, and U.S. Pat. No. 10,702,208 (“Apparatus and Method for Electroencephalographic Examination”) issued Jul. 7, 2020, both of which are hereby incorporated by reference in their entireties. Therefore, it is difficult to create a one-size-fits-all prediction model. Instead, individualized models need to be created for each patient, taking into account their unique physiology and response to different sedatives.

Most patients under general anesthesia are exposed to oversedation while others are still at risk of intraoperative awareness and postoperative recall, which is proven harmful. Processed EEG monitoring can be useful in preventing intraoperative awareness with explicit recall compared with clinical signs but it is not compared with anesthetic concentration alarms. To minimize inappropriate sedation, processed EEG-based techniques currently in use, like BIS Index, Narcotrend, and Sedline, have been recommended and widely used to assess intra-operative sedation levels. However, multiple large, randomized trials have shown that such techniques provide a limited value as individual BIS scores demonstrate wide variability, making it difficult to assess the depth of sedation. It does not reduce the rate of intraoperative awareness. Even more, a study showed no difference in the BIS index between those who had intraoperative awareness and those who did not.

A major weakness of processed EEG-based techniques currently in use for intraoperative sedation monitoring is its population-based approach, but there is a large inter-individual variation of the EEG at a given clinically assessed sedation level. Weaknesses of the processed EEG-based techniques can include 1) its inability to reliably determine the patients' consciousness or unconsciousness; 2) large inter-individual variability of BIS index values at the loss of consciousness (LOC); and 3) each sedative has its own EEG signature at a given level of sedation, but multiple drugs affecting sedation level are usually concurrently administered to a patient under general anesthesia. Even more importantly, no statistically significant correlation has been identified between the BIS index value and EEG at a clinically assessed LOC. Hence, individualized sedation monitoring is needed to overcome the challenge.

This disclosure presents a novel methodology that utilizes an artificial intelligence (AI)-patient interactive approach and sedation profile of the individual patient AI models (from online learning) during the process of falling asleep to determine the level of sedation (continuous monitoring and measurement). With this technique, a patient's EEG profile corresponds to a given sedation level, and therefore, sedation monitoring using an AI model is less likely to be impacted by inter-individual variation, which is the limitation in existing anesthesia monitoring systems. Patient-self-defined LOC and AI-assisted EEG processing can be utilized to monitor the sedation level of the patients under general anesthesia in a personalized manner. This novel sedation monitoring system can reduce the incidence of both undersedation and oversedation and improve the quality of patient care.

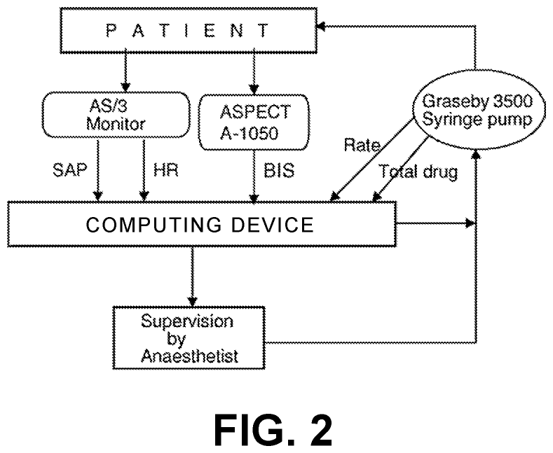

Self-referenced machine learning can be extremely beneficial for personalized sedation. By using data from the same patient, the machine learning algorithm can learn to adjust itself to avoid the influence of large heterogeneity among different patients and better assess an individual's sedation levels during general anesthesia or sedation. Referring to FIG. 2, shown is an example of a closed-loop control system for anesthesia delivery to a patient. As shown in FIG. 1, the EEG and BIS of the patient can be monitored using monitoring apparatus. Other variable, like systemic arterial blood pressure (SAP) and heart rate (HR) can also be monitored. An application for anesthesia depth monitoring and control can be executed by a computing device and used to control the anesthesia provided to the patient based upon, for example, the monitored patient variables and/or dispensing rates and amounts. The self-referenced-dynamic algorithm can use, for example, the EEG at different sedation levels obtained during induction to determine the ongoing sedation level and assess instantaneously the sedation level and the trend towards to away from the target (or optimal) sedation level. The information is used to adjust the sedative dose or rate or use the automatic closed-loop drug delivery system to maintain an optimal sedation level. The overall operation of the system can be supervised by an anesthetist. In some implementations, the system can include a camera, video, or other optical devices for monitoring the state of the patient.

During induction, the level of consciousness declines from fully awake to LOC with a corresponding EEG profile at each sedation level. The AI can be trained to remember the EEG profile at each sedation level up to LOC for that specific individual and analyze the prospectively collected EEG profiles to determine if the level of sedation for the specific individual is at the target level and if the trend of the ongoing EEG profile is approaching or moving away from the target sedation level. Therefore, the prediction of the monitoring sedation level is not interfered by inter-individual variability and is also independent of an agent-specific EEG signature (as long as the drugs used for the index anesthesia are not changed, even dosing is allowed to change).

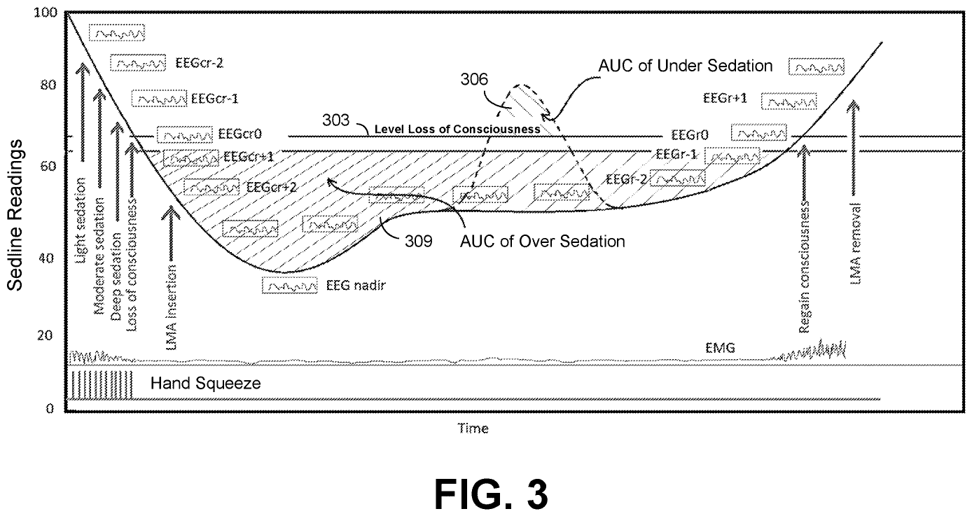

For example, EEG information can be obtained during the induction of anesthesia and used to train the machine learning algorithm to achieve personalized control. FIG. 3 illustrates an example of EEG profiles and EEG readings vs. time during general anesthesia. After initiation of sedation, the patient passes through light, moderate and heavy (or deep) sedation until loss of consciousness is reached (illustrated as line 303). Undersedation is defined as the sedation level above the level of the loss of consciousness (e.g., 306), and oversedation (e.g., 309) is below the sedation level of one minute (EEGcr+1) after the loss of consciousness 303 occurs.

A baseline EEG can be recorded for one minute with the eye open and then closed for one minute before induction of anesthesia. After the baseline EEG is recorded, anesthesia is induced by, e.g., intravenous (IV) infusion of propofol as a total of intravenous anesthesia (TIVA) at a dose of, e.g., 0.2 to 0.6 mg/kg/minute, which induces LOC of adult human beings in about 5 minutes. The slow induction allows for observation of the dynamic change in EEG profile corresponding to different levels of sedation.

During induction of anesthesia, the patient can be asked with a pre-recorded audio command via a headphone in a usual tone to squeeze his or her left hand every 5 seconds. The response can be monitored in a variety of ways (e.g., with a pressure sensitive grip, with a video camera, by the care team, etc.). The EEG is continuously recorded as a time-series of data. The first time the patient fails to respond to the verbal command, the patient can be considered to be in a LOC state, and the corresponding profile from the EEG is labeled as EEG critical (EEGcr0) as shown in FIG. 3. The EEG collected at 2 minutes and 1 minute before LOC are identified as EEGcr−2 and EEGcr−1, respectively, and at 1 and 2 minutes after LOC are identified as EEGcr+1 and EEGcr+2, respectively. Two minutes after LOC, the airway can be secured with either a supraglottic airway or an end tracheal tube, and propofol infusion continues. The EEG monitoring is continued during the procedure. The infusion rate of propofol can be adjusted by the care team and/or system to maintain the target sedation level. Upon the completion of the surgery, propofol infusion is terminated, and emergence starts. Monitoring the EEG can continue until the patient regains his or her consciousness and the supraglottic airway or end tracheal tube is removed.

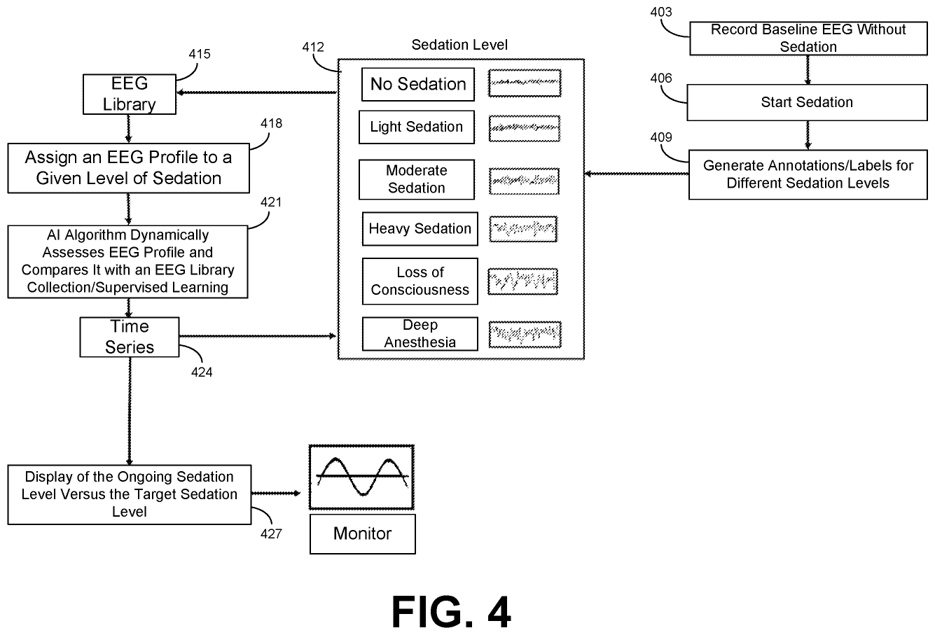

FIG. 4 illustrates an example of the monitoring flow. Beginning at 403, the baseline EEG data is recorded and stored before initiation of sedation at 406 and the different levels of sedation that are recorded and stored can be labeled as no sedation, light sedation, moderate sedation, and heavy sedation at 409. Once the patient reaches loss of consciousness (LOC), the acquired time-series EEG data is accordingly labeled at 412. Deep anesthesia is also recorded, stored and labeled after the loss of consciousness at 412. The acquired EEG data allows for individualized training of the AI model for the monitored patient, but also allows the training to account for the current condition of the patient. In this way, the anesthesia depth monitoring can be customized to account for any changes the patient at the time of the procedure.

The recorded time-series EEG data can be segregated into data frames (e.g., 5-second frames) as a unit for analysis. Adjacent data frames can include portions that overlap. For example, a one-second waveform can overlap the two immediately adjacent frames of 5 seconds. Using the labeled EEG data frames, a library of EEG frames can be established at 415. The AI system can be assigned (or trained) to remember each of the EEG frames associated with a given sedation level at 418 and constantly compares the profiles with the ongoing EEG data at 421 to determine the level of sedation relative to the target sedation level, for example, LOC level (EEGcr0). The sedation level estimated from ongoing EEG and the target level of sedation can be displayed on a monitor at 424 to guide the care team and/or control adjustment of the infusion rate of propofol (and/or bolus).

The described approach enables the care team to determine the ongoing sedation level, stay close to the target sedation level, minimize the probability of intraoperative awareness, and eliminate post-operative recalls. This can lead to minimizing oversedation and undersedation, reducing complications associated with undersedation and oversedation, shortening emergence time, and possibly the post-operative care unit stay. In some implementations, the identified level of sedation level can be used to control the anesthesia to maintain a desired level of sedation. For example, the amount of drug(s) delivered to the patient by a syringe pump or other metering device can be controlled based on comparing the estimated and target sedation levels.

The EEG data (or other monitored data) can be captured as a time-series and processed for use by the machine learning algorithm. Initially, the data can be preprocessed to remove noise or artifacts that may be present. As a part of the self-referenced algorithm, the data preprocessing can be accomplished in an online manner. This means that filtering, normalization, and feature selection can all be made as part of the learning process, rather than done beforehand. This allows the learning process to be more flexible since it can adapt to changes in the data more easily.

As discussed, the time-series EEG data is segmented into smaller segments in order to process and learn from them on-the-fly. This can happen automatically in real-time so that data is divided up into smaller chunks as needed at one or more resolutions. In this way, the AI model receives content that can be tailored specifically for different stages, including awake, light sedation, moderate sedation, deep sedation, and loss of consciousness, to be efficient and robust to changes in the data.

Feature extraction from raw EEG signal data can be conducted for the self-referenced machine-learning model. By extracting features (e.g., Fourier transforms, wavelet transforms, and time-frequency analysis) from the EEG data in real-time and aggregating them in dynamic data segments, more personalized learning can be supported as the features extracted can be tailored to individual patients.

For the machine learning, a residual neural network where connections between units are not only made forward but also backward and to the same layer can be trained. This creates a so-called “residual” connection, which helps the network learn complex functions. Super-resolution can be introduced as a process of adding samples near the boundary to improve the resolution of EEG segments next to stage changes. The residual neural network will be trained incrementally on short segments of data, in contrast to traditional neural networks, which must be trained on all data before they can be used. This allows the AI model to adapt to new data when its distribution changes over time. The training process includes data from the different sedation levels from fully awake to complete LOC, and can comprise data from deep anesthesia after LOC. As discussed, the different levels of sedation are achieved by slowly administering sedatives, either inhalational agents or intravenous sedatives.

While the training observes awake to complete loss of consciousness, the AI model can be tested on unseen EEG data to predict different sedation levels (including awake). Since the model is trained on the fly, it allows new EEG signals to be classified in real-time and reflect the sedation level at any given time point. The instantaneously measured sedation level can be output to control a control module. The instantaneously measured sedation level is used to guide the clinician to adjust the delivery of the sedatives, inhalational or intravenous sedatives, and to achieve and maintain the target sedation level. The performance of the algorithm can be evaluated in terms of accuracy and the area under the receiver operating characteristics curve (AUROC).

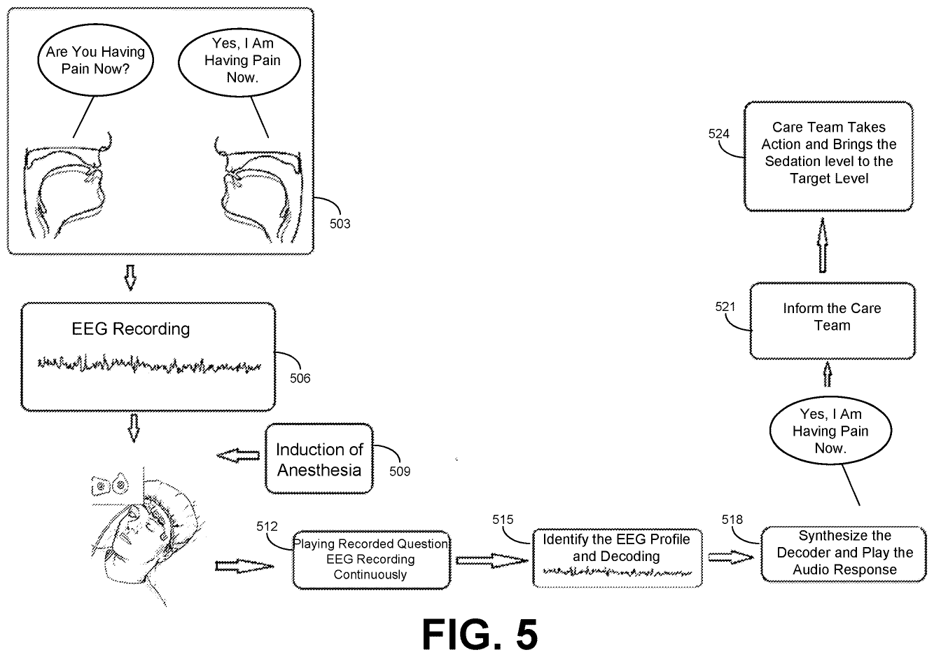

In some embodiments, a brain-computer-interface can be used in monitoring sedation depth of the patient whose muscle is intentionally paralyzed under general anesthesia. Even if the muscle is paralyzed, the cortical processing of articulation is still intact in those patients. Therefore, their EEG profile in responding to a speech stimulation should be nearly identical to that before they are paralyzed. FIG. 5 illustrates an example of a procedure that can be used. The EEG in responding to an articulation of a sentence can be recorded before the initiation of anesthesia (and in some cases during induction of anesthesia). For instance, at 503 the care team can ask the patient to articulate, “yes, I am having pain now” or “no, I do not have pain” after the care team asks the patient, “are you having pain now?” The EEG profile can be recorded during the question and the answer and stored at 506. The questions and answers can be repeated a few times to acquire baseline information. Induction of anesthesia can be carried out at 509. In some cases, the questions and answers can be repeated during this process and the EEG signals recorded.

Then the question can be played intermittently during the procedure with the patient under anesthesia and EEG signal can be continuously recorded at 512. The EEG profile obtained during the response to the answer can be used to train the AI, and AI can constantly compare the EEG profile with the prospectively recorded EEG signal in real time. Once the corresponding EEG signal matches the EEG in response to the answer in real-time, the patient can be considered awake and indicating having or having no pain. The EEG signal can be decoded at 515 and synthesized with, e.g., pre-recorded sentences and transformed into an audio response 518 that can be used to inform care team at 521. Therefore, the care team can be instantaneously made aware that the patient is awake and, if she or he is in pain, can promptly take action to keep the patient at the target sedation level at 524.

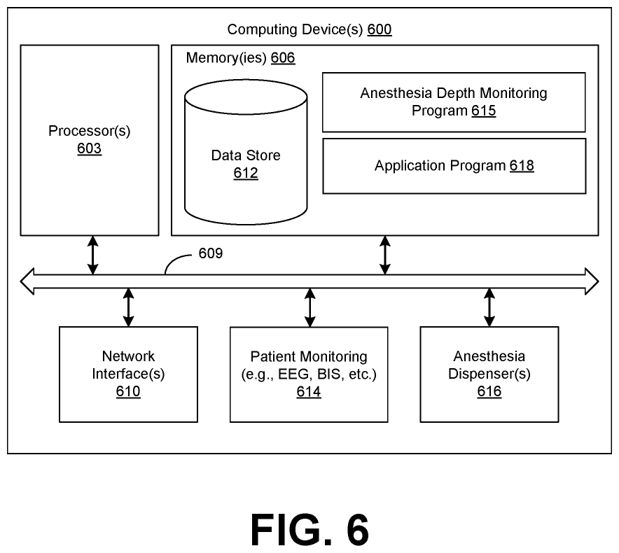

With reference to FIG. 6, shown is a schematic block diagram of a computing device 600 that can be utilized for monitoring and control of anesthesia depth. In some embodiments, among others, the computing device 600 may represent a mobile device (e.g., a smartphone, tablet, computer, etc.). Each computing device 600 includes at least one processor circuit, for example, having a processor 603 and a memory 606, both of which are coupled to a local interface 609. To this end, each computing device 600 may comprise, for example, at least one server computer or like device. The local interface 609 may include, for example, a data bus with an accompanying address/control bus or other bus structure, as can be appreciated.

In some embodiments, the computing device 600 can include one or more network interfaces 610. The network interface 610 may comprise, for example, a wireless transmitter, a wireless transceiver, and a wireless receiver. As discussed above, the network interface 610 can communicate with a remote computing device using a Bluetooth protocol. As one skilled in the art can appreciate, other wireless protocols may be used in the various embodiments of the present disclosure. The computing device 600 can also interface with patient monitoring (e.g., EEG, BIS, etc.) devices 614 and anesthesia dispenser(s) 616 for monitoring and/or controlling anesthesia depth.

Stored in the memory 606 are both data and several components that are executable by processor 603. In particular, stored in memory 606 and executable by the processor 603 are an anesthesia depth monitoring program 615, application program 618, and potentially other applications. Also stored in the memory 606 may be a data store 612 and other data. In addition, an operating system may be stored in memory 606 and executable by processor 603.

It is understood that there may be other applications that are stored in memory 606 and are executable by processor 603 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in memory 606 and are executable by processor 603. In this respect, the term “executable” means a program file that is in a form that can ultimately be run by the processor 603. Examples of executable programs may be, for example, a compiled program that can be translated into machine code in a format that can be loaded into a random access portion of the memory 606 and run by the processor 603, source code that may be expressed in proper format such as object code that is capable of being loaded into a random access portion of the memory 606 and executed by the processor 603, or source code that may be interpreted by another executable program to generate instructions in a random access portion of the memory 606 to be executed by the processor 603, etc. An executable program may be stored in any portion or component of the memory 606 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 606 is defined herein as including both volatile and nonvolatile memory and data storage components. Volatile components are those that do not retain data values upon loss of power. Nonvolatile components are those that retain data upon a loss of power. Thus, the memory 606 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 603 may represent multiple processors 603 and/or multiple processor cores and the memory 606 may represent multiple memories 606 that operate in parallel processing circuits, respectively. In such a case, the local interface 609 may be an appropriate network that facilitates communication between any two of the multiple processors 603, between any processor 603 and any of the memories 606, or between any two of the memories 606, etc. The local interface 609 may comprise additional systems designed to coordinate this communication, including, for example, performing load balancing. The processor 603 may be of electrical or of some other available construction.

Although the anesthesia depth monitoring program 615 and the application program 618, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the anesthesia depth monitoring program 615 and the application program 618, that comprises software or code can be embodied in any non-transitory computer-readable medium for use by or in connection with an instruction execution system such as, for example, a processor 603 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the anesthesia depth monitoring program 615 and the application program 618, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 600, or in multiple computing devices in the same computing environment. Additionally, it is understood that terms such as “application,” “service,” “system,” “engine,” “module,” and so on may be interchangeable and are not intended to be limiting.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that do not negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity and, thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A method for anesthesia depth monitoring, comprising:
obtaining baseline EEG data of a subject prior to sedation;
obtaining time-series EEG data of the subject after initiation of sedation;
identifying loss of consciousness (LOC) of the subject;
generating profiles segmented from the time-series EEG data, the profiles corresponding to the LOC of the subject and a plurality of sedation levels of the subject before and after LOC, where the plurality of sedation levels comprise a target sedation level for the subject;
training an artificial intelligence (AI) sedation model associated with the subject using the generated profiles, the AI sedation model trained to determine a sedation level of the subject based upon a profile segmented from the time-series EEG data of the subject;
determining a current sedation level of the subject based upon a current profile segmented from the time-series EEG data of the subject; and
providing an indication of a comparison of the current sedation level with the target sedation level.

2. The method of claim 1, wherein the plurality of sedation levels comprise undersedation and oversedation levels.

3. The method of claim 2, wherein the undersedation levels comprise light sedation, moderate sedation, and heavy sedation levels.

4. The method of claim 1, wherein the identification of the LOC comprises monitoring the subject's responsiveness to a rhythmic stimulus, wherein the LOC is identified based on a lack of a response to the rhythmic stimulus.

5. The method of claim 4, wherein the rhythmic stimulus is a pre-recorded audio command.

6. The method of claim 4, wherein monitoring responsiveness comprises detecting a physical response or an EEG profile provided by the subject in response to the rhythmic stimulus.

7. The method of claim 1, wherein the baseline EEG data comprises EEG data obtained with the subject's eyes open and closed.

8. The method of claim 1, wherein the plurality of sedation levels comprise sedation levels at two minutes before LOC, one minute before LOC, one minute after LOC, and two minutes after LOC.

9. The method of claim 1, wherein the AI sedation model comprises a residual neural network.

10. A system for anesthesia depth monitoring, comprising:
monitoring apparatus configured to provide EEG data of a subject; and
a computing device comprising processing circuitry configured to:
obtain baseline EEG data of the subject from the monitoring apparatus prior to sedation and time-series EEG data of the subject after initiation of sedation;
generate profiles segmented from the time-series EEG data, the profiles corresponding to an identified loss of consciousness (LOC) of the subject and a plurality of sedation levels of the subject before and after the LOC, where the plurality of sedation levels comprise a target sedation level for the subject;
train an artificial intelligence (AI) sedation model associated with the subject using the generated profiles, the AI sedation model trained to determine a sedation level of the subject based upon a profile segmented from the time-series EEG data of the subject;
determine a current sedation level of the subject based upon a current profile segmented from the time-series EEG data of the subject; and
provide an indication of a comparison of the current sedation level with the target sedation level.

11. The system of claim 10, wherein the plurality of sedation levels comprise undersedation and oversedation levels.

12. The system of claim 10, wherein the plurality of sedation levels comprise sedation levels at two minutes before LOC, one minute before LOC, one minute after LOC, and two minutes after LOC.

13. The system of claim 10, wherein the identification of the LOC comprises monitoring the subject's responsiveness to a rhythmic stimulus, wherein the LOC is identified based on a lack of a response to the rhythmic stimulus.

14. The system of claim 13, wherein the rhythmic stimulus is a pre-recorded audio command provided by the computing device.

15. The system of claim 13, wherein monitoring responsiveness comprises detecting a physical response provided by the subject in response to the rhythmic stimulus.

16. The system of claim 15, comprising a sensor configured to detect grip pressure applied in response to the rhythmic stimulus.

17. The system of claim 13, wherein monitoring responsiveness comprises detecting an EEG profile generated by the subject in response to the rhythmic stimulus.

18. The system of claim 13, wherein the processing circuitry is further configured to detect an EEG profile generated by the subject in response to a verbal command, the EEG profile indicating a current condition of the subject.

19. The system of claim 10, wherein providing the indication of the comparison comprises providing the current sedation level with the target sedation level for display on a monitor.

20. The system of claim 10, wherein the processing circuitry is configured to provide an adjustment to the sedation based upon the comparison of the current sedation level with the target sedation level.
